(19) United States
(12) Patent Application Publication
(10) Pub. No.: US 2023/0080495 A1
(43) Pub. Date: Mar. 16, 2023

(54) ELECTRONIC DEVICE, METHOD FOR SELECTING ALGORITHM, AND RECORDING MEDIUM

(71) Applicant: CASIO COMPUTER CO., LTD., Tokyo (JP)

(72) Inventors: Shuto OIKAWA, Tokyo (JP); Takeshi MIYAKE, Tokyo (JP); Tatsuyoshi OMURA, Tokyo (JP); Keiichi NOMURA, Uenohara-shi (JP); Tomoo KASUO, Tokyo (JP)

(73) Assignee: CASIO COMPUTER CO., LTD., Tokyo (JP)

(21) Appl. No.: 17/882,022

(22) Filed: Aug. 5, 2022

(30) Foreign Application Priority Data
Sep. 14, 2021 (JP) 2021-149753

(51) Int. Cl. A61B 5/00; A61B 5/11; A61B 5/024

(52) U.S. Cl. CPC A61B 5/7264; A61B 5/7275; A61B 5/1118; A61B 5/02438; A61B 5/681

(57) ABSTRACT

An electronic device includes a biometric detection value acquirer to acquire a biometric detection value for calculation of biometric information of a wearer of the electronic device and a processor, in which the processor estimates a change trend of information related to an action detail of the wearer and, based on the estimated change trend of information related to an action detail, selects an algorithm to calculate the biometric information from the biometric detection value.

### CROSS-REFERENCE TO RELATED APPLICATION

This application claims the benefit of Japanese Patent Application No. 2021-149753, filed on Sep. 14, 2021, the entire disclosure of which is incorporated by reference herein.

### FIELD

This application relates generally to an electronic device, a method for selecting an algorithm, and a recording medium.

### BACKGROUND

Recent years, electronic devices that are worn on the body and are capable of measuring biometric information, such as a pulse rate, using a sensor, such as an optical sensor, have been developed. While such an electronic device enables biometric information of a user (wearer) to be easily measured, there have been some cases where, while the user is performing intense exercise, noise components are likely to be contained in acquisition values (sensor values) from the sensor and accuracy of measurement values of the biometric information is reduced. In order to solve this problem, in, for example, Unexamined Japanese Patent Application Publication No. 2017-148312, a sensor information processing device and the like that changes, according to an exercise state of the user, an algorithm to calculate biometric information from sensor values is disclosed.

The prior art as disclosed in Unexamined Japanese Patent Application Publication No. 2017-148312 is configured to suppress noise components contained in sensor values by detecting an exercise state of the user and changing a pulse rate detection algorithm, based on characteristics of the sensor values estimated from the exercise state. In practice, however, characteristics of sensor values do not necessarily change in complete synchronization with change in the exercise state of the user, and there often exists a temporal gap between timing at which the characteristics of the sensor values change and timing at which the exercise state changes. However, in the prior art, existence of such a temporal gap has not been taken into consideration.

### SUMMARY

An electronic device according to one aspect of the present disclosure includes:

a biometric detection value acquirer to acquire a biometric detection value for calculation of biometric information of a wearer of the electronic device; and

a processor,

wherein the processor

estimates a change trend of information related to an action detail of the wearer, and

based on the estimated change trend of information related to an action detail, selects an algorithm to calculate the biometric information from the biometric detection value.

A method for selecting an algorithm in an electronic device according to one aspect of the present disclosure is a method for selecting an algorithm in an electronic device including a biometric detection value acquirer to acquire a biometric detection value for calculation of biometric information of a wearer and a processor of the present disclosure includes the processor:

estimating a change trend of information related to an action detail of the wearer; and

based on the estimated change trend of information related to an action detail, selecting an algorithm to calculate the biometric information from the biometric detection value.

A non-transitory computer-readable recording medium according to one aspect of the present disclosure is a non-transitory computer-readable recording medium storing a program executable by a processor of an electronic device, the electronic device including a biometric detection value acquirer to acquire a biometric detection value for calculation of biometric information of a wearer and the processor,

wherein the processor, in accordance with the program,

estimates a change trend of information related to an action detail of the wearer, and

based on the estimated change trend of information related to an action detail, selects an algorithm to calculate the biometric information from the biometric detection value.

### BRIEF DESCRIPTION OF THE DRAWINGS

A more complete understanding of this application can be obtained when the following detailed description is considered in conjunction with the following drawings, in which:

FIG. 1 is a block diagram illustrating an example of a functional configuration of an electronic device according to an embodiment;

FIG. 2 is a diagram illustrating an example of an external appearance of the electronic device when viewed from the front side;

FIG. 3 is a diagram illustrating an example of an external appearance of the electronic device when viewed from the back side;

FIG. 4 is a diagram illustrating an example of an external appearance of an electronic device including a pulse rate display that displays a pulse rate by a hand;

FIG. 5 is a diagram illustrating an example of an external appearance of an electronic device including a pulse rate display that displays pulse rates by a graph;

FIG. 6 is an example of a flowchart of pulse rate display processing according to the embodiment;

FIG. 7 is a first portion of an example of a flowchart of algorithm selection processing according to the embodiment;

FIG. 8 is a second portion of the example of the flowchart of the algorithm selection processing according to the embodiment;

FIG. 9 is a third portion of the example of the flowchart of the algorithm selection processing according to the embodiment;

FIG. 10 is a fourth portion of the example of the flowchart of the algorithm selection processing according to the embodiment;

FIG. 11 is a fifth portion of the example of the flowchart of the algorithm selection processing according to the embodiment;

FIG. 12 is a diagram illustrating an example of change in pulse rates;

FIG. 13 is an example of a flowchart of profile generation processing according to the embodiment;

FIG. 14 is a diagram illustrating a display example in the pulse rate display that displays a plurality of candidates of pulse rates;

FIG. 15 is an example of a flowchart of pulse rate correction processing according to the embodiment; and

FIG. 16 is a diagram illustrating a display example in the pulse rate display when the pulse rate correction processing is performed.

### DETAILED DESCRIPTION

An electronic device and the like according to an embodiment is described with reference to the drawings. Note that, in the drawings, the same or equivalent constituent elements are designated by the same reference numerals.

### Embodiments

An electronic device according to the embodiment is a wristwatch-type device that is capable of measuring a pulse rate of a user by being worn on a wrist of the user, and is, for example, a smartwatch.

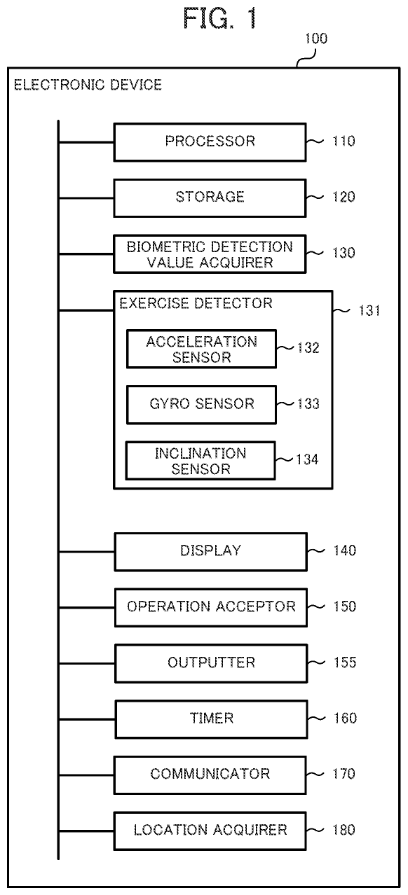

An electronic device 100 according to the embodiment includes a processor 110, a storage 120, a biometric detection value acquirer 130, an exercise detector 131, a display 140, an operation acceptor 150, an outputter 155, a timer 160, a communicator 170, and a location acquirer 180, as illustrated in FIG. 1.

The processor 110 includes a processor, such as a central processing unit (CPU). The processor 110 executes pulse rate display processing and the like, which are described later, by executing programs stored in the storage 120. In addition, the processor 110 has a capability of performing multi-thread processing and is thus capable of executing a plurality of processes in parallel.

The storage 120 stores programs that the processor 110 executes and data necessary for the execution of the programs. The storage 120 may include a random access memory (RAM), a read only memory (ROM), a flash memory, or the like, but not limited thereto. Note that the storage 120 may be installed inside the processor 110.

The biometric detection value acquirer 130 includes a light emitting diode (LED) and a photodiode (PD) as a pulse wave sensor. The biometric detection value acquirer 130 receives, by the PD, light that was generated when light having been emitted from the LED toward a living body was reflected inside the living body, and detects a pulse wave, based on temporal change in received light intensity. The processor 110 acquires, as biometric detection values, values to which the received light intensity at the PD is analog-to-digital (AD) converted (AD values), and calculates a pulse rate, based on temporal change in the AD values.

The exercise detector 131 includes an acceleration sensor 132, a gyro sensor 133, and an inclination sensor 134 and acquires detection values (exercise detection values) of the respective sensors. However, as long as the exercise detector 131 includes at least one sensor to detect an exercise state of the user (for example, the acceleration sensor 132), the exercise detector 131 does not have to include the other sensors. In addition, in order to detect an exercise state of the user, the exercise detector 131 may include a sensor other than the acceleration sensor 132, the gyro sensor 133, and the inclination sensor 134 (such as a geomagnetic sensor and a pressure sensor). For example, by detecting the amount of change in altitude by the pressure sensor, it is possible to detect the user ascending or descending a slope.

The acceleration sensor 132 is a triaxial acceleration sensor that detects motion in the three axial directions orthogonal to one another. For example, when the user wearing the electronic device 100 moves, the processor 110 can acquire detection values representing in what direction and with how much acceleration the user has moved, from the acceleration sensor 132.

The gyro sensor 133 is an angular velocity sensor that detects angular velocity of rotation. For example, when the user wearing the electronic device 100 turns the body, the processor 110 can acquire detection values representing in what direction and with how much angular velocity the body has rotated, from the gyro sensor 133.

The inclination sensor 134 measures an inclination angle of an object, based on the force of gravity. For example, when the electronic device 100 is tilted, the processor 110 can detect the electronic device 100 inclining, by the inclination sensor 134.

The display 140 includes physical hands and a display device, such as a liquid crystal display and an organic electro-luminescence (EL) display. The display 140 displays a pulse rate measured by the biometric detection value acquirer 130, a time timed by the timer 160, and the like. Note that the display 140 may include an analog time display including physical hands (a second hand, a minute hand, and an hour hand), a day wheel, a motor driver, a motor, and a wheel train mechanism. In addition, the display 140 may, instead of including a physical analog time display, perform analog time display by displaying hands on a display device, such as a liquid crystal display.

The operation acceptor 150 is a user interface including a crown, a push-button switch, and the like and accepts operation input from the user. The processor 110 is capable of acquiring what operation input the user has performed, based on detection results, such as a rotational state of the crown and a pressed state of the switch of the operation acceptor 150. Note that, when the electronic device 100 includes a touch panel integrated with the display 140, the touch panel also serves as the operation acceptor 150 and accepts a tap operation and the like performed by the user.

The outputter 155 includes a speaker and outputs a voice announcement and sound effects. Note that the electronic device 100 may include, instead of a speaker or in addition to a speaker, an LED (light emitter) or a vibrator as the outputter 155.

The timer 160 times the time that the electronic device 100 displays on the display 140. The timer 160 also has a function of a timer to measure a specified time period. Note that the timer 160 may be configured by software that changes a value to be stored at a predetermined address in the storage 120 every predetermined time period (for example, one second) or configured by dedicated hardware. Note also that the timer 160 may be installed inside the processor 110.

The communicator 170 is a communication interface for the electronic device 100 to perform data communication with an external device (such as a smartphone, a tablet, a personal computer (PC), and another smartwatch) and acquire information from the Internet. The communicator 170 may include a wireless communication interface for performing communication using, for example, Bluetooth (registered trademark) or a wireless local area network (LAN), but not limited thereto.

The location acquirer 180 receives satellite signals transmitted from global positioning system (GPS) satellites and thereby acquires a current location of the electronic device 100. Since the location acquirer 180 cannot receive a satellite signal indoors, the processor 110 is also capable of determining whether the current location is outdoor or indoor, based on whether or not the location acquirer 180 can receive satellite signals.

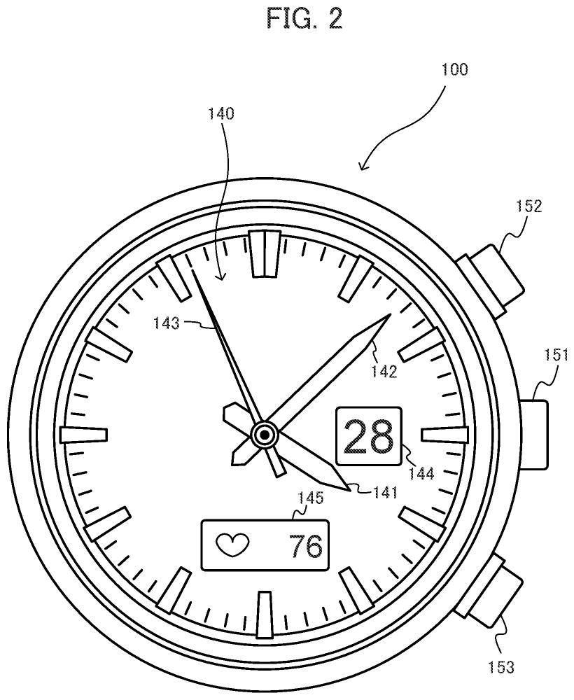

In appearance, the electronic device 100 includes an hour hand 141, a minute hand 142, a second hand 143, a day wheel 144, and a pulse rate display 145 as the display 140 on the front side, as illustrated in FIG. 2. The electronic device 100 displays a time, a date, and a pulse rate of the user by the hour hand 141, minute hand 142, and second hand 143, the day wheel 144, and the pulse rate display 145, respectively.

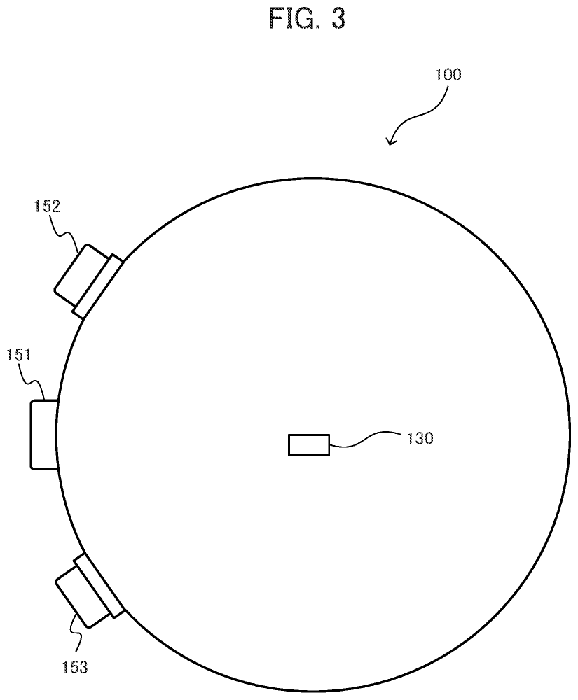

In addition, the electronic device 100 includes a crown 151 and push-button switches 152 and 153 on the lateral side, as illustrated in FIG. 2, and accepts operation performed thereon by the user. The electronic device 100 also includes the biometric detection value acquirer 130 on the back side, as illustrated in FIG. 3.

The electronic device 100 calculates a pulse rate of the user from AD values acquired by the biometric detection value acquirer 130, using a pulse rate measurement algorithm. In this configuration, since the AD values are values acquired from the biometric detection value acquirer 130 (pulse wave sensor), the AD values are also referred to as biometric detection values (sensor values). In the present embodiment, as the pulse rate measurement algorithm, four types of algorithms, namely an algorithm for low-level trend, an algorithm for upward trend, an algorithm for high-level trend, and an algorithm for downward trend, are provided according to a change trend of the pulse rate.

In any one of the pulse rate measurement algorithms, the processor 110 basically calculates the number of pulsepulses per minute, based on temporal increase and decrease in AD values acquired from the biometric detection value acquirer 130 and calculates a pulse rate, based on a moving average of the number of pulsepulses. Since noise components due to body motion or the like are added to the temporal increase and decrease in AD values, when the processor 110 calculates the number of pulsepulses per minute, the processor 110 performs frequency analysis (Fourier transformation or the like) of a waveform of the AD values and thereby acquires frequency components. Since, in the frequency components, components other than the pulse rate, such as noise components due to body motion or the like and harmonic components, are included, the processor 110 generally cannot determine a pulse rate uniquely.

Therefore, the pulse rate measurement algorithm calculates candidates of the pulse rate in conjunction with likelihoods (probabilities of the pulse rates) at a predetermined time interval (for example, one second interval). A pulse rate with the highest likelihood is displayed at the time interval on the pulse rate display 145. For example, when the pulse rate measurement algorithm calculates three candidates of the pulse rate and a candidate 1 is 60 beats per minute (bpm) with a likelihood of 50%, a candidate 2 is 90 bpm with a likelihood of 40%, and a candidate 3 is 30 bpm with a likelihood of 10%, 60 is displayed on the pulse rate display 145 as a pulse rate.

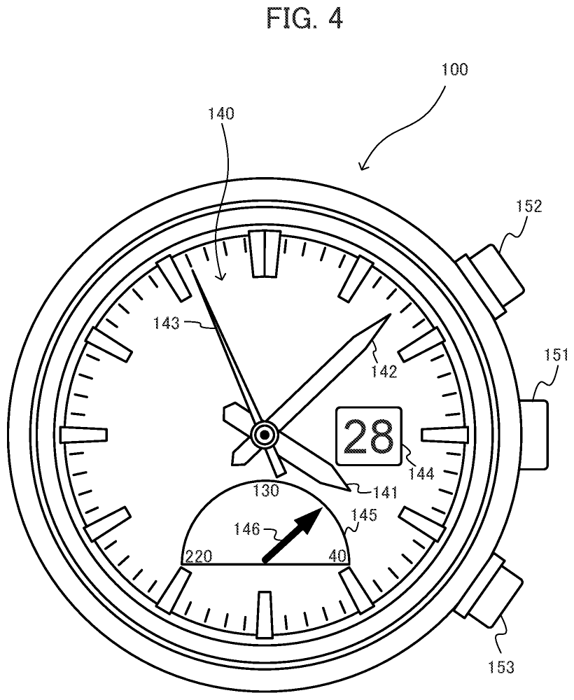

In addition, the display of the pulse rate on the pulse rate display 145 is not limited to digital display. The electronic device 100 may include a pulse rate display 145 to display a pulse rate in an analog manner by a hand 146, as illustrated in FIG. 4. Displaying a pulse rate by the hand 146 enables the user to visually grasp the magnitude of the pulse rate through the angle of the hand 146 without recognizing a figure.

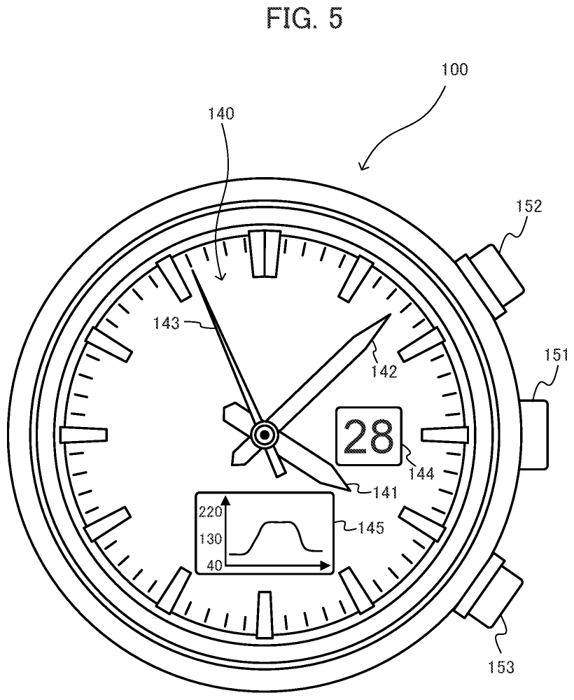

In addition, the display of the pulse rate on the pulse rate display 145 is not limited to digital display and analog display. The electronic device 100 may display the pulse rate using a graph on the pulse rate display 145, as illustrated in FIG. 5. Displaying the pulse rate using a graph enables the user to easily recognize temporal change in the pulse rate. Note that the electronic device 100 may send pulse rate information to another device, such as a smartphone and a PC, using the communicator 170 and cause the other device to display the pulse rate using a graph.

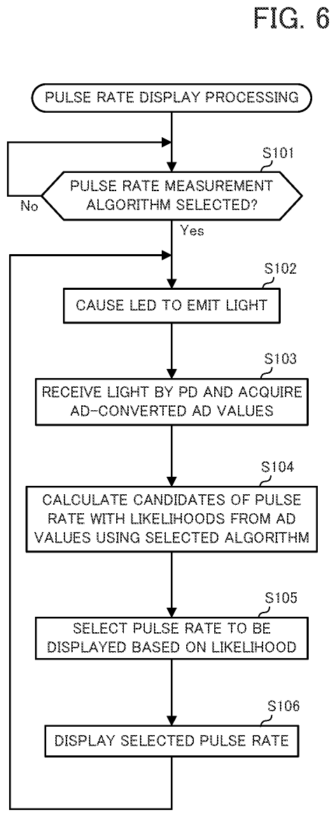

Next, the pulse rate display processing, which is processing of the electronic device 100 displaying a pulse rate, is described with reference to FIG. 6. Note, however, that, since a pulse rate measurement algorithm, which is used at the time of calculating a pulse rate in the pulse rate display processing, is selected in algorithm selection processing, which is described later, execution of the pulse rate display processing requires the algorithm selection processing to be executed in parallel. For example, when the user instructs, through the operation acceptor 150, the electronic device 100 to display a pulse rate, the pulse rate display processing and the algorithm selection processing are started. In addition, the electronic device 100 may be configured to, when being started up, start the execution of the pulse rate display processing and the algorithm selection processing in parallel with other processing.

When the pulse rate display processing is started, the processor 110 first determines whether or not a pulse rate measurement algorithm has been selected by the algorithm selection processing (step S101). When no pulse rate measurement algorithm has been selected (step S101; No), the processor 110 returns to step S101. However, since, as described later, as soon as the algorithm selection processing is started, the algorithm for low-level trend is selected as the pulse rate measurement algorithm, the determination in step S101 normally results in Yes immediately.

When a pulse rate measurement algorithm has been selected (step S101; Yes), the processor 110 causes the LED of the biometric detection value acquirer 130 to emit light (step S102). Light having been emitted from the LED and reflected by the living body is received by the PD of the biometric detection value acquirer 130, and the processor 110 acquires AD values to which received light intensity at the PD has been converted by the AD converter (step S103).

Next, the processor 110 calculates candidates of the pulse rate of the user in conjunction with likelihoods of the candidates from the AD values, using the currently selected pulse rate measurement algorithm (step S104). Next, the processor 110 selects a pulse rate to be displayed from among the candidates of the pulse rate, based on the calculated likelihoods (step S105). In step S104, the processor 110 normally selects a pulse rate with the highest likelihood.

Next, the processor 110 displays a pulse rate selected in step S105 on the pulse rate display 145 (step S106) and returns to step S102.

In the algorithm selection processing, which is executed in parallel with the execution of the above-described pulse rate display processing, the processor 110 estimates an action detail (exercise state) of the user, estimates a change trend of information (pulse rate) related to the estimated action detail, based on the estimated action detail, and selects an algorithm (pulse rate measurement algorithm) for calculating biometric information (pulse rate) from biometric detection values (AD values), based on the estimated change trend. That is, the processor 110 selects a most appropriate pulse rate measurement algorithm from among the four types of algorithms, which are the algorithm for low-level trend, the algorithm for upward trend, the algorithm for high-level trend, and the algorithm for downward trend, according to a change trend of the pulse rate. The processor 110 selecting a pulse rate measurement algorithm from among the four types of algorithms enables measurement precision of a pulse rate to be improved.

The algorithm for low-level trend is an algorithm that is selected when the pulse rate is estimated to have a trend of being stable at a comparatively low value. In this algorithm, the processor 110 outputs, as a pulse rate, a moving average for a first standard time (a comparatively long time, for example, 10 seconds) of the number of pulses per minute calculated from AD values. When the user is at rest or in a normal condition (when the user is not exercising), this algorithm is basically selected. The algorithm for low-level trend is, by using a moving average for a comparatively long time, capable of reducing influence of noise and the like and thereby enables a pulse rate when the user is not exercising to be output more accurately.

The algorithm for upward trend is an algorithm that is selected when the pulse rate is estimated to have an upward trend. In this algorithm, the processor 110 outputs, as a pulse rate, a moving average for a second standard time (a comparatively short time, for example, 5 seconds) of the number of pulses per minute calculated from AD values. In the algorithm for upward trend, setting a time period used for calculating a moving average to a comparatively short time causes follow-up performance to an increase in the pulse rate to be improved.

In the algorithm for upward trend, the processor 110 also calculates a moving average for the first standard time of the number of pulses per minute calculated from the AD values. Since, while this algorithm is being selected, the pulse rate is expected to be in an upward trend, when the pulse rate represented by a moving average for the second standard time indicates a downward trend, this algorithm reduces the rate of decrease by outputting a moving average for the first standard time as a pulse rate. This configuration enables the pulse rate to be prevented from decreasing due to influence of noise and the like as much as possible.

The algorithm for high-level trend is an algorithm that is selected when the pulse rate is estimated to have a trend of being stable at a comparatively high value. In this algorithm, the processor 110 outputs, as a pulse rate, a moving average for the second standard time of the number of pulses per minute calculated from AD values. In this algorithm, setting a time period used for calculating a moving average to a comparatively short time improves follow-up performance to a change in the pulse rate. This is because, when the user is exercising, the pulse rate sometimes further increases and sometimes decreases depending on change in the amount of exercise of the user.

The algorithm for downward trend is an algorithm that is selected when the pulse rate is estimated to have a downward trend. In this algorithm, the processor 110 outputs, as a pulse rate, a moving average for the second standard time of the number of pulses per minute calculated from AD values. In the algorithm for downward trend, setting a time period used for calculating a moving average to a comparatively short time improves follow-up performance to a decrease in the pulse rate.

In the algorithm for downward trend, the processor 110 also calculates a moving average for the first standard time of the number of pulses per minute calculated from the AD values. Since, while this algorithm is being selected, the pulse rate is expected to be in a downward trend, when the pulse rate represented by the moving average for the second standard time indicates an upward trend, this algorithm reduces the rate of increase by outputting a moving average for the first standard time as a pulse rate. This configuration enables the pulse rate to be prevented from increasing due to influence of noise and the like as much as possible.

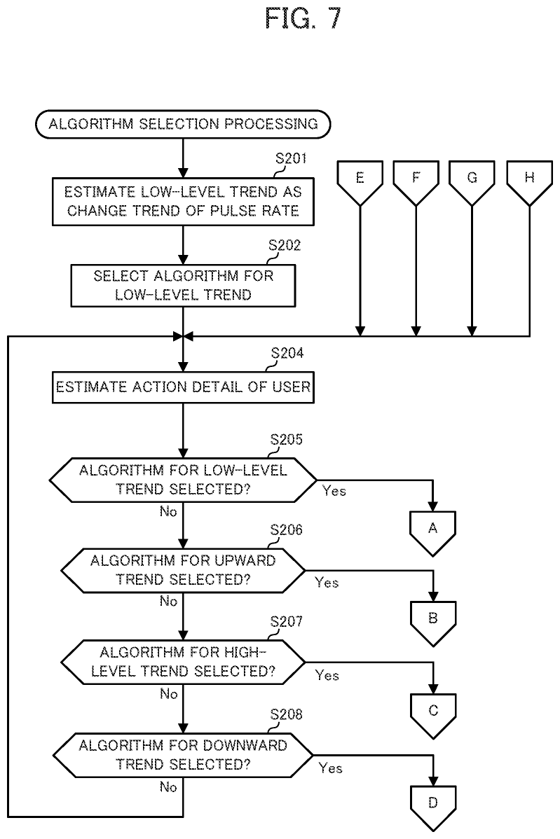

The algorithm selection processing, which is processing of the electronic device 100 selecting a pulse rate measurement algorithm, is described with reference to FIGS. 7 to 11. This processing is, as with the above-described pulse rate display processing, started in response to an instruction by the user or when the electronic device 100 is started up.

When the algorithm selection processing is started, the processor 110 first estimates the change trend of the pulse rate to be a “low-level trend” (step S201) and selects the algorithm for low-level trend as the pulse rate measurement algorithm (step S202). This processing being performed causes measurement of the pulse rate by the above-described pulse rate display processing to be started.

Although a value that the processor 110 estimates as the change trend of the pulse rate in the algorithm selection processing (an estimated value of the change trend of the pulse rate) is the “low-level trend”, the “upward trend”, the “high-level trend”, or the “downward trend”, there is a high possibility that the change trend of the pulse rate of the user in normal conditions is the “low-level trend”. Thus, the processor 110 sets an initial value of the estimated value of the change trend of the pulse rate to the “low-level trend” and selects the algorithm for low-level trend as an initial setting of the pulse rate measurement algorithm in steps S201 and S202. In practice, however, there is a possibility that the change trend of the pulse rate at this time is not the “low-level trend”. However, the processor 110 is to repeatedly perform processing of estimating an action detail of the user and estimating a change trend of the pulse rate in the succeeding process (a loop from step S204 onward). Therefore, even when the processor 110 incorrectly estimated a change trend of the pulse rate in the beginning, the processor 110 increasingly becomes able to accurately estimate a change trend of the pulse rate.

Note that, when this processing is actually coded into a program, the change trend itself does not have to be treated directly as a value. For example, the change trends “low-level trend”, “upward trend”, “high-level trend”, and “downward trend” may be represented by integer values of 1, 2, 3, and 4, respectively. When the processor 110, using these values, assigns a result of estimation in which the processor 110 estimated a change trend of the pulse rate (an estimated value of the change trend) to a variable V, the processor 110 assigns a value of 1 to the variable V as an initial value in step S201 and subsequently assigns values of 2, 4, 3, 4, 2, 2, and 1 to the variable V in steps S222, S242, S245, S262, S265, S282, and S285, which are described later, respectively.

In addition, when the user is supposed to wear the electronic device 100 for a comparatively long period of time (for example, all day long), the processor 110 may perform processing of determining whether or not the pulse rate is stable at a comparatively low value (step S203) after the processing in step S202. In this case, when the pulse rate is not stable at a comparatively low value, the processor 110 waits in step S203 until the pulse rate is stabilized at a low value. This is because, when the user wears the electronic device 100 for a long period of time, the processor 110 can automatically acquire a pulse rate when the pulse rate is stable at a comparatively low value, based on pulse rates measured during the period of time. In addition, while waiting in step S203, the processor 110 may display on the display 140 or output by voice from the outputter 155 a message, such as “Please rest quietly”, in order to ask the user to rest.

Next, the processor 110 estimates an action detail of the user (step S204). Although any method can be used as a method for the processor 110 to estimate an action detail of the user in step S204, examples of such a method include estimation based on a detection result of the exercise detector 131, estimation based on action patterns (action history) of the user in the past, estimation based on an action schedule that the user has registered, and estimation using the location acquirer 180. In addition, the processor 110 may estimate an action detail of the user by combining a plurality of methods from among the above-described estimation methods.

The estimation based on a detection result of the exercise detector 131 is a method of estimating an action detail of the user, based on detection values (exercise detection values) detected by the sensor that the exercise detector 131 includes. For example, the processor 110, using a known action estimation method, such as machine learning, estimates an action detail that the user is currently performing (such as, at rest, walking, and during exercise like brisk walking, low-speed running (light running), high-speed running (sprint), pedaling a bicycle, or fitness exercise), based on detection values detected by the exercise detector 131. The processor 110 is capable of not only estimating an action detail of the user in real time by estimating an action, based on exercise detection values but also improving precision of action estimation by choosing a sensor used for the action estimation as needed.

The estimation based on action patterns (action history) of the user in the past is a method of storing action details that the processor 110 estimated in the past, in conjunction with information indicating dates and times in the storage 120 as an action history and, by using the action history, estimating an action detail of the user. For example, the processor 110, using information of the current time (and day of the week) as a key, extracts an action detail performed in the same time zone (on the same day of the week) from within the action history and estimates that the user must be performing the extracted action detail currently. In the case of using this method, the storage 120 includes an action history storage to store an action history of the user. The processor 110, by performing estimation based on the action history, is capable of using past estimation results effectively.

The estimation based on an action schedule that the user has registered is a method of estimating an action detail of the user, using information of an action schedule that the user has registered in the storage 120 in advance. For example, when the user has registered, as an action schedule, an action schedule such as “to do light running from 20:00 to 21:00 on weekdays”, the processor 110, using information of the current time (and day of the week) as a key, extracts an action detail performed in the same time zone (on the same day of the week) from among the action schedules and estimates that the user must be performing the extracted action detail currently. In the case of using this method, the storage 120 includes an action schedule storage to store action schedules of the user. The processor 110, by performing estimation based on the action schedules, is capable of estimating an action accurately when the user acts in accordance with an action schedule that the user himself/herself has registered.

The estimation using the location acquirer 180 is a method of estimating an action detail of the user, based on location information acquired using the location acquirer 180. For example, when the processor 110, using the location acquirer 180, detects that the user has gone outdoors, the processor 110 estimates that the user is to start exercise. In addition, when a location acquired by the location acquirer 180 is the location of a facility for exercise, the processor 110 may estimate, as the current action detail of the user, that the user is currently exercising. The processor 110, by performing estimation using the location acquirer 180, is capable of estimating an action that is considered to be reasonable, based on the current location of the user.

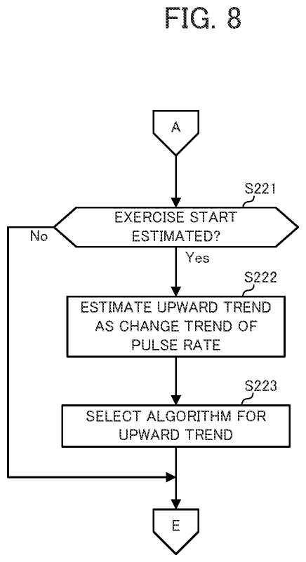

Returning to FIG. 7, the processor 110 determines whether or not the algorithm for low-level trend has been selected as the pulse rate measurement algorithm (step S205). When the algorithm for low-level trend has been selected (step S205; Yes), the processor 110 proceeds to FIG. 8 and determines whether or not the processor 110 estimated “exercise start” as an estimation result of the action detail of the user in step S204 (step S221). In this processing, estimating the “exercise start” does not require the type of exercise to be distinguished, and, when it is estimated that some type of exercise has been started, the determination in step S221 results in Yes. However, when the estimated action detail is the “rest” or the “walking”, the action is not considered as exercise, and the determination in step S221 results in No.

When the processor 110 did not estimate the “exercise start” as an estimation result of the action detail of the user (step S221; No), the processor 110 returns to step S204 in FIG. 7 and performs estimation of an action detail of the user again.

When the processor 110 estimated the “exercise start” as an estimation result of the action detail of the user (step S221; Yes), the processor 110 estimates the “upward trend” as the change trend of the pulse rate (step S222). The processor 110 then selects the algorithm for upward trend as the pulse rate measurement algorithm (step S223) and returns to step S204 in FIG. 7.

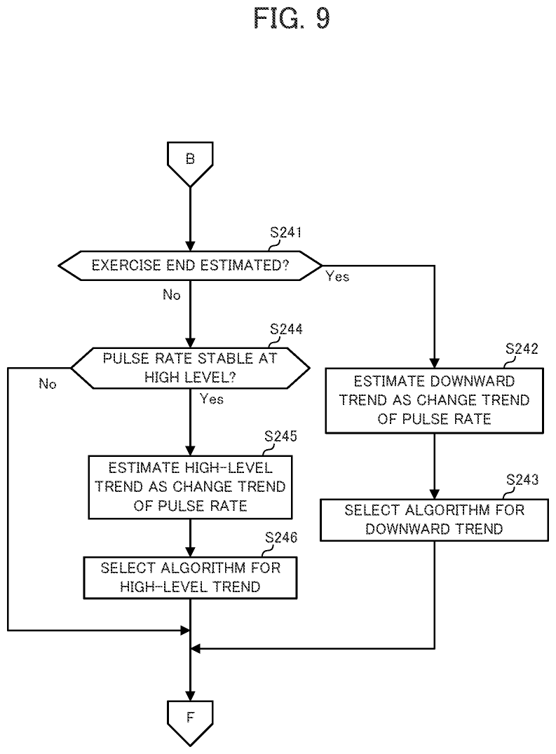

In contrast, when, in step S205 in FIG. 7, the algorithm for low-level trend has not been selected as the pulse rate measurement algorithm (step S205; No), the processor 110 determines whether or not the algorithm for upward trend has been selected as the pulse rate measurement algorithm (step S206). When the algorithm for upward trend has been selected (step S206; Yes), the processor 110 proceeds to FIG. 9 and determines whether or not the processor 110 estimated “exercise end” as an estimation result of the action detail of the user in step S204 (step S241). In this processing, estimating the “exercise end” does not require the type of exercise to be distinguished, and, when it is estimated that some type of exercise has been finished (for example, an action detail estimated in step S204 in the current loop is the “rest” or the “walking”), the determination in step S241 results in Yes.

When the processor 110 estimated the “exercise end” as an estimation result of the action detail of the user (step S241; Yes), the processor 110 estimates the “downward trend” as the change trend of the pulse rate (step S242). The processor 110 then selects the algorithm for downward trend as the pulse rate measurement algorithm (step S243) and returns to step S204 in FIG. 7.

In contrast, when, in the determination in step S241, it is determined that the processor 110 did not estimate the “exercise end” as an estimation result of the action detail of the user (step S241; No), the processor 110 determines whether or not the pulse rate is stable at a comparatively high value (step S244). Specifically, when the pulse rate calculated in the pulse rate display processing, which is being executed in parallel, is greater than or equal to a high-level standard value (for example, 100 bpm) and variation in the pulse rate is less than or equal to a standard variation value (for example, ±5 bpm/min), the processor 110 determines that the pulse rate is stable at a comparatively high value.

When the pulse rate is not stable at a comparatively high value (step S244; No), the processor 110 returns to step S204 in FIG. 7.

When the pulse rate is stable at a comparatively high value (step S244; Yes), the processor 110 estimates the “high-level trend” as the change trend of the pulse rate (step S245). The processor 110 then selects the algorithm for high-level trend as the pulse rate measurement algorithm (step S246) and returns to step S204 in FIG. 7.

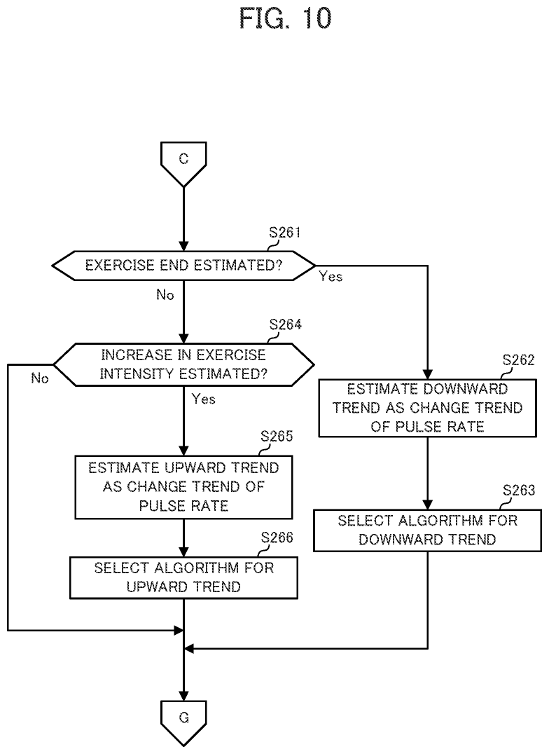

In contrast, when, in step S206 in FIG. 7, the algorithm for upward trend has not been selected as the pulse rate measurement algorithm (step S206; No), the processor 110 determines whether or not the algorithm for high-level trend has been selected as the pulse rate measurement algorithm (step S207). When the algorithm for high-level trend has been selected (step S207; Yes), the processor 110 proceeds to FIG. 10 and determines whether or not the processor 110 estimated the “exercise end” as an estimation result of the action detail of the user in step S204 (step S261).

When the processor 110 estimated the “exercise end” as an estimation result of the action detail of the user (step S261; Yes), the processor 110 estimates the “downward trend” as the change trend of the pulse rate (step S262). The processor 110 then selects the algorithm for downward trend as the pulse rate measurement algorithm (step S263) and returns to step S204 in FIG. 7.

In contrast, when, in the determination in step S261, it is determined that the processor 110 did not estimate the “exercise end” as an estimation result of the action detail of the user (step S261; No), the processor 110 determines whether or not the processor 110 estimated an increase in exercise intensity as an estimation result of the action detail of the user in step S204 (step S264). Specifically, the processor 110 determines that the processor 110 estimated an increase in the exercise intensity when movement velocity detected by the exercise detector 131 had increased by a standard rate of increase (for example, 10%), when an increase in the exercise intensity was able to be estimated from the estimated action detail (for example, when the exercise had changed from the “low-speed running” to the “high-speed running”), when the amount of change in altitude detected by the pressure sensor had increased by the standard amount of altitude change (for example, 20%), or the like.

When the processor 110 did not estimate an increase in the exercise intensity (step S264; No), the processor 110 returns to step S204 in FIG. 7.

When the processor 110 estimated an increase in the exercise intensity (step S264; Yes), the processor 110 estimates the “upward trend” as the change trend of the pulse rate (step S265). The processor 110 then selects the algorithm for upward trend as the pulse rate measurement algorithm (step S266) and returns to step S204 in FIG. 7.

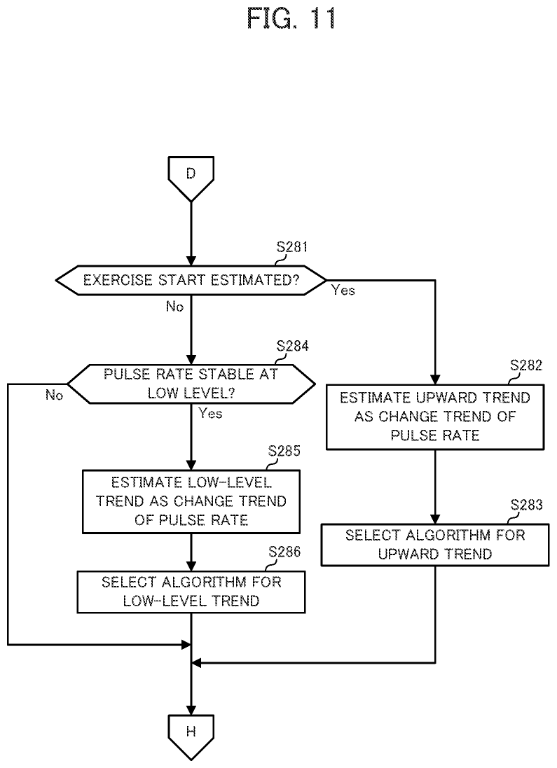

In contrast, when, in step S207 in FIG. 7, the algorithm for high-level trend has not been selected as the pulse rate measurement algorithm (step S207; No), the processor 110 determines whether or not the algorithm for downward trend has been selected as the pulse rate measurement algorithm (step S208). When the algorithm for downward trend has been selected (step S208; Yes), the processor 110 proceeds to FIG. 11 and determines whether or not the processor 110 estimated the “exercise start” as an estimation result of the action detail of the user in step S204 (step S281).

When the processor 110 estimated the “exercise start” as an estimation result of the action detail of the user (step S281; Yes), the processor 110 estimates the “upward trend” as the change trend of the pulse rate (step S282). The processor 110 then selects the algorithm for upward trend as the pulse rate measurement algorithm (step S283) and returns to step S204 in FIG. 7.

In contrast, when, in the determination in step S281, it is determined that the processor 110 did not estimate the “exercise start” as an estimation result of the action detail of the user (step S281; No), the processor 110 determines whether or not the pulse rate is stable at a comparatively low value (step S284). Specifically, when the pulse rate calculated in the pulse rate display processing, which is being executed in parallel, is less than or equal to a low-level standard value (for example, 100 bpm) and variation in the pulse rate is less than or equal to the standard variation value (for example, ±5 bpm/min), the processor 110 determines that the pulse rate is stable at a comparatively low value.

When the pulse rate is not stable at a comparatively low value (step S284; No), the processor 110 returns to step S204 in FIG. 7.

When the pulse rate is stable at a comparatively low value (step S284; Yes), the processor 110 estimates the “low-level trend” as the change trend of the pulse rate (step S285). The processor 110 then selects the algorithm for low-level trend as the pulse rate measurement algorithm (step S286) and returns to step S204 in FIG. 7.

In contrast, when, in step S208 in FIG. 7, the algorithm for downward trend has not been selected as the pulse rate measurement algorithm (step S208; No), the processor 110 returns to step S204.

Since, through the pulse rate display processing and the algorithm selection processing described above, the electronic device 100 estimates a change trend of the pulse rate of the user and selects a pulse rate measurement algorithm suitable for the estimated change trend, the electronic device 100 is capable of selecting an appropriate algorithm before a change in the pulse rate actually occurs (including a timing at which the pulse rate changes). Since the electronic device 100 is, by measuring the pulse rate using an algorithm selected in this way, capable of following change in the pulse rate appropriately, the electronic device 100 is capable of improving accuracy of a measurement value of the pulse rate. In addition, in the algorithm selection processing, the electronic device 100 is, by estimating an action detail of the user, capable of estimating a change trend of the pulse rate appropriately.

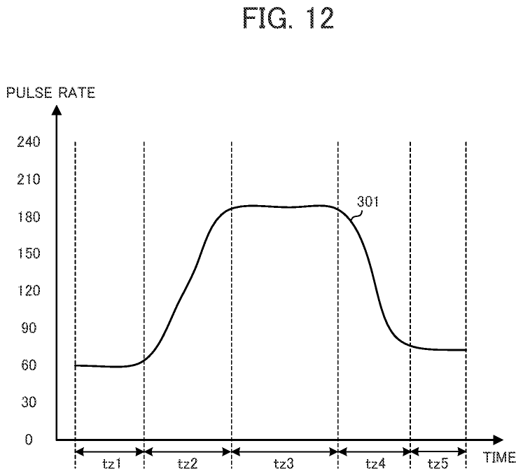

For example, when the pulse rate of a user changes as illustrated by a solid line 301 in FIG. 12, the algorithm for low-level trend, the algorithm for upward trend, the algorithm for high-level trend, the algorithm for downward trend, and the algorithm for low-level trend are selected in time zones tz1, tz2, tz3, tz4, and tz5, respectively, as a pulse rate measurement algorithm. Thus, in the time zones tz1 and tz5, the use of a moving average for a comparatively long time enables the electronic device 100 to measure stable pulse rates with less error. In addition, in the time zones tz2, tz3, and tz4, the use of a moving average for a comparatively short time enables the electronic device 100 to follow change in the pulse rate. In addition, the use of an algorithm capable of easily following increase in the pulse rate and an algorithm capable of easily following decrease in the pulse rate in the time zones tz2 and tz4, respectively, enables the electronic device 100 to measure a pulse rate with less error.

Note that, in the above-described algorithm selection processing, the processor 110 estimates an action detail of the user in step S204 and, based on a result of the estimation, estimates a change trend of the pulse rate. However, the processor 110 may estimate a change trend of the pulse rate, based on an exercise detection value that the exercise detector 131 acquired, an action schedule stored in the action schedule storage, a location that the location acquirer acquired, and the like, without estimating an action detail of the user.

In addition, since it can be considered that, in the above-described algorithm selection processing, the processor 110 selecting one of the algorithm for low-level trend, the algorithm for upward trend, the algorithm for high-level trend, and the algorithm for downward trend in steps S202, S223, S243, S246, S263, S266, S283, and S286 indicates that the processor 110 estimates, as the change trend of the pulse rate, one of the “low-level trend”, the “upward trend”, the “high-level trend”, and the “downward trend”, the processor 110 does not have to perform the processing in steps S201, S222, S242, S245, S262, S265, S282, and S285.

Variation 1

In the above-described embodiment, in the algorithm for upward trend and the algorithm for downward trend, a moving average for a comparatively short time of the number of pulses is normally output as a pulse rate and, when the calculated value is determined to be an abnormal value, a moving average for a comparatively long time of the number of pulses is output as a pulse rate. It is when the pulse rate indicates a downward trend in the algorithm for upward trend and the pulse rate indicates an upward trend in the algorithm for downward trend that a calculated value is determined as an abnormal value. However, the determination of an abnormal value is not limited to the above-described determination, and an abnormal value may be determined based on data of the pulse rate accumulated in the past. As Variation 1, an embodiment in which a user profile of the pulse rate is generated and an abnormal value is determined using the user profile is described.

In Variation 1, the processor 110 accumulates calculated pulse rates in the storage 120 and, when some amount of data (for example, pulse rate data of ten exercises in the past) have been accumulated, generates a user profile, based on pulse rate data having been accumulated up to that time, and determines a value that does not fit the generated user profile as an abnormal value.

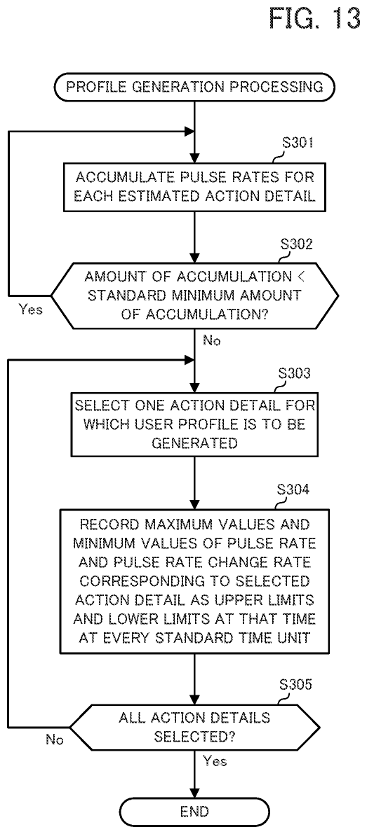

Profile generation processing in which the processor 110 generates a user profile is described with reference to FIG. 13. The profile generation processing may be configured to be started by an instruction of the user or to be started in parallel with other processing when the electronic device 100 is started up.

When the profile generation processing is started, the processor 110 first accumulates pulse rates calculated in the above-described pulse rate display processing in the storage 120 with respect to each action detail of the user estimated in the algorithm selection processing (step S301). For example, when the user has performed the “brisk walking” for 10 minutes, the “low-speed running” for 5 minutes, and the “brisk walking” for 3 minutes, in the storage 120, data of the pulse rate for initial 10 minutes are accumulated as pulse rate data corresponding to an action detail “first brisk walking”, data of the pulse rate for succeeding 5 minutes are accumulated as pulse rate data corresponding to an action detail “first low-speed running”, and data of the pulse rate for further succeeding 3 minutes are accumulated as pulse rate data corresponding to an action detail “second brisk walking”.

Next, the processor 110 determines whether or not the amount of data accumulated in step S301 is less than the standard minimum amount of accumulation (for example, when an action detail (for example, the “brisk walking”) continuing for longer than or equal to a standard accumulation time (for example, one minute) is counted as one exercise, the amount of accumulation equivalent to 10 exercises) (step S302). When the amount of accumulated data is less than the standard minimum amount of accumulation (step S302; Yes), the processor 110 returns to step S301 and continues accumulation of pulse rates.

When the amount of accumulated data is greater than or equal to the standard minimum amount of accumulation (step S302; No), the processor 110 selects one action detail corresponding to accumulated pulse rate data in the storage 120 (for example, the “brisk walking”) (step S303).

Next, the processor 110 extracts a plurality of pieces of pulse rate data corresponding to the selected action detail at every standard time unit (for example, one second) in a chronological order and stores, in the storage 120, a maximum value and a minimum value among pulse rates at each time and a maximum value and a minimum value among pulse rate change rates at each time as upper limits and lower limits of the pulse rate and the pulse rate change rate at the time (step S304).

For example, it is assumed that pulse rates at every second from the start time (0 seconds) of “first brisk walking” to 2 seconds later are 60, 61, and 63, pulse rates at every second from the start time (0 seconds) of “second brisk walking” to 2 seconds later are 70, 69, and 72, and pulse rates at every second from the start time (0 seconds) of “third brisk walking” to 2 seconds later are 71, 65, and 60. In addition, when a pulse rate change rate at a time (t) is defined as a value obtained by subtracting a pulse rate at the time (t) from a pulse rate at a time (t+1), pulse rate change rates at every second from the start time (0 seconds) of the “first brisk walking” to 1 second later are 1 and 2, pulse rate change rates at every second from the start time (0 seconds) of the “second brisk walking” to 1 second later are −1 and 3, and pulse rate change rates at every second from the start time (0 seconds) of the “third brisk walking” to 1 second later are −6 and −5.

In step S304, the processor 110 records 71 (the pulse rate in the third brisk walking) and 60 (the pulse rate in the first brisk walking) as an upper limit and a lower limit of the pulse rate, respectively, and 1 (the pulse rate change rate in the first brisk walking) and −6 (the pulse rate change rate in the third brisk walking) as an upper limit and a lower limit of the pulse rate change rate, respectively, at the start time (0 seconds) and also records 69 (the pulse rate in the second brisk walking) and 61 (the pulse rate in the first brisk walking) as an upper limit and a lower limit of the pulse rate, respectively, and 3 (the pulse rate change rate in the second brisk walking) and −5 (the pulse rate change rate in the third brisk walking) as an upper limit and a lower limit of the pulse rate change rate, respectively, at 1 second after the start time.

In step S304, the upper limits and the lower limits of the pulse rate and the pulse rate change rate at each time of the action detail selected in step S303 are recorded in the storage 120 in this way.

Next, the processor 110 determines whether or not the processor 110 has finished selecting all the action details corresponding to the pulse rate data accumulated in the storage 120 in step S303 having been executed up to that time (step S305). When the processor 110 has not finished selecting all the action details (step S305; No), the processor 110 returns to step S303. In this way, the upper limits and the lower limits of the pulse rate and the pulse rate change rate at each time for each action detail are recorded in the storage 120 in this way. The upper limits and the lower limits of the pulse rate and the pulse rate change rate at each time for each action detail are a user profile.

When the processor 110 has finished selecting all the action details corresponding to the pulse rate data accumulated in the storage 120 (step S305; Yes), the processor 110 terminates the profile generation processing.

In the algorithm for upward trend, when a pulse rate or a pulse rate change rate calculated using a moving average for the second standard time (comparatively short time) exceeds the corresponding upper limit in the user profile generated in the above-described profile generation processing, the processor 110 outputs a moving average for the first standard time (comparatively long time) as a pulse rate. This configuration enables the pulse rate to be prevented from increasing too rapidly.

In addition, in the algorithm for downward trend, when a pulse rate or a pulse rate change rate calculated using a moving average for the second standard time (comparatively short time) falls below the corresponding lower limit in the user profile generated in the above-described profile generation processing, the processor 110 outputs a moving average for the first standard time (comparatively long time) as a pulse rate. This configuration enables the pulse rate to be prevented from decreasing too rapidly.

In Variation 1, as described above, the use of data of the pulse rate of the user in the past (user profile) enables accuracy of the pulse rate to be further improved.

Variation 2

In the above-described embodiment and Variation 1, the processor 110 regards a pulse rate with the highest likelihood among calculated pulse rates as a correct pulse rate and displays the pulse rate on the display 140. In practice, however, a pulse rate with the highest likelihood is not necessarily a correct pulse rate. As Variation 2, an embodiment that is capable of correcting a pulse rate that the processor 110 calculates, using a reference device capable of outputting an accurate pulse rate or guided by the user is described.

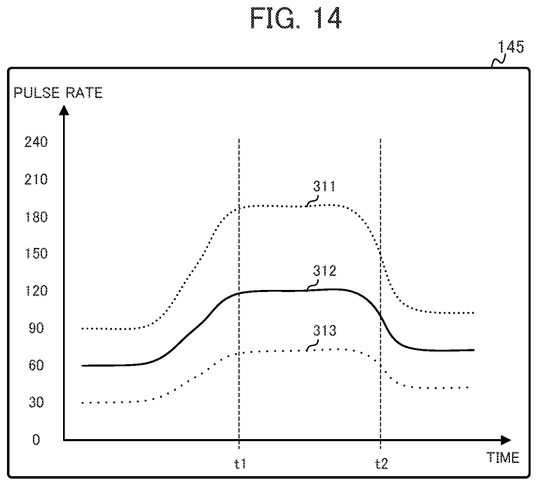

In the above-described embodiment, as illustrated in FIG. 5, for example, only pulse rates with the maximum likelihood are displayed on the pulse rate display 145. In contrast, the electronic device 100 according to Variation 2 also displays pulse rates the likelihood of which is not the maximum on the pulse rate display 145, as illustrated in, for example, FIG. 14. In FIG. 14, as an example, a graph of pulse rates with a likelihood of 50%, a graph of pulse rates with a likelihood of 40%, and a graph of pulse rates with a likelihood of 10% are illustrated by a solid line 312, a dotted line 311, and a dotted line 313, respectively. In such a case, in the configuration of the above-described embodiment, which is different from that of Variation 2, only the solid line 312 was displayed on the pulse rate display 145 as a graph of the pulse rate.

It is now assumed that, in this example, at and after a time t1, pulse rates illustrated by the solid line 312 are not correct and pulse rates illustrated by the dotted line 311 are correct. In this case, at the time t1, for example, the user informing the electronic device 100 according to Variation 2 of correct pulse rates being indicated by the dotted line 311 enables the electronic device 100 to display the correct pulse rates.

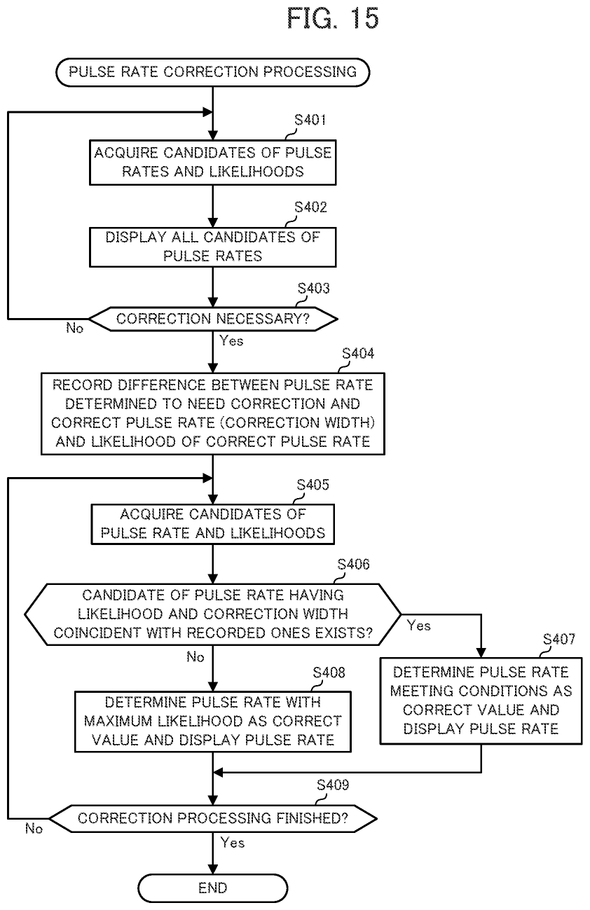

Pulse rate correction processing of performing such correction of a pulse rate is described with reference to FIG. 15. The pulse rate correction processing is started when the user instructs the electronic device 100 to execute the pulse rate correction processing through the operation acceptor 150. However, since, in order to execute the pulse rate correction processing, the above-described pulse rate display processing and algorithm selection processing need to be executed in parallel, when the pulse rate display processing and the algorithm selection processing are not being executed, the pulse rate display processing and the algorithm selection processing are started before the pulse rate correction processing is started and subsequently the pulse rate correction processing is started.

When the pulse rate correction processing is started, the processor 110 acquires candidates of pulse rates and likelihoods thereof calculated in the pulse rate display processing, which is being executed in parallel (step S401). Next, the processor 110 displays all the acquired candidates of pulse rates on the pulse rate display 145 (step S402). However, when the number of candidates is too many to display all of the candidates, the processor 110 may be configured to display a predetermined number of candidates in descending order of likelihood (for example, only the top three candidates). In addition, in order to make a magnitude relationship in likelihood among the candidates clearly understandable, the processor 110 may be configured to, for example, display a pulse rate candidate with the maximum likelihood by a solid line and pulse rate candidates other than the pulse rate candidate with the maximum likelihood by dotted lines.

Next, the processor 110 determines whether or not correction of a pulse rate is necessary (step S403). For example, the processor 110 determines that correction of a pulse rate is necessary when correction of the pulse rate is instructed by a user operation (when a graph of pulse rates other than the pulse rates with the maximum likelihood is tapped on the pulse rate display 145, or the like), when error between the pulse rate and a pulse rate calculated by the reference device is greater than or equal to a standard error (for example, 10 bpm), or the like.

When the processor 110 does not determine that correction of a pulse rate is necessary (step S403; No), the processor 110 returns to step S401. When the processor 110 determines that correction of a pulse rate is necessary (step S403; Yes), the processor 110 records a difference between the pulse rate determined to need to be corrected and a correct pulse rate (correction width) and the likelihood of the correct pulse rate in the storage 120 (step S404).

Next, as with the processing in step S401, the processor 110 acquires candidates of the pulse rate and likelihoods thereof calculated in the pulse rate display processing, which is being executed in parallel (step S405). Next, the processor 110 determines whether or not a candidate of the pulse rate the likelihood and correction width of which coincide with the likelihood and the correction width recorded in step S404 exists among the acquired candidates of the pulse rate (step S406). In this processing, “coinciding with the likelihood and the correction width” means that error in likelihood is less than or equal to a standard likelihood error (for example, 10%) and error in correction width is less than or equal to a standard correction width error (for example, 10%).

As an example, both the standard likelihood error and the standard correction width error are set to 10%, and it is assumed that, for example, a pulse rate of 60 bpm with a likelihood of 50% is determined to need to be corrected in step S403 and a correct pulse rate is 90 bpm and the likelihood thereof is 40% at the time of correction. In this case, “a likelihood of 40% and a correction width of 30 bpm” are recorded in step S404. It is also assumed that, in step S405, a candidate having a pulse rate of 62 bpm with a likelihood of 52%, a candidate having a pulse rate of 91 bpm with a likelihood of 42%, and a candidate having a pulse rate of 35 bpm with a likelihood of 6% are acquired as candidates of the pulse rate. Then, in step S406, the processor 110 determines whether or not a candidate of the pulse rate that has a likelihood within the recorded likelihood plus/minus the standard likelihood error (36% to 44%) and a difference between which and a pulse rate (62 bpm) with the maximum likelihood is a correction width within the recorded correction width plus/minus the standard correction width error (27 bpm to 33 bpm) exists. Since, in this example, a candidate having “a pulse rate of 91 bpm with a likelihood of 42%” that satisfied the above-described conditions exists, the determination in step S406 results in Yes.

When a candidate having a likelihood and a correction width that coincide with the likelihood and the correction width recorded in step S404 exists among the candidates of the pulse rate acquired in step S405 (step S406; Yes), the processor 110 determines that the pulse rate that meets the conditions in step S406 as a correct value and displays the pulse rate on the pulse rate display 145 (step S407). For example, the processor 110 displays pulse rates determined to be correct values by a solid line and displays pulse rates other than the correct pulse rates by a dotted line.

In contrast, when no candidate having a likelihood and a correction width that coincide with the likelihood and the correction width recorded in step S404 exists (step S406; No), the processor 110 determines that a pulse rate with the maximum likelihood among the candidates of the pulse rate acquired in step S405 is a correct value and displays the pulse rate on the pulse rate display 145 (step S408). For example, the processor 110 displays pulse rates with the maximum likelihood by a solid line and displays pulse rates other than the pulse rates with the maximum likelihood by a dotted line.

Next, the processor 110 determines whether or not to terminate the correction processing (step S409). When, for example, termination of the correction processing is instructed by a user operation, the processor 110 determines to terminate the correction processing. When an action detail of the user that was estimated in the algorithm selection processing, which was being executed in parallel, has changed (for example, when the “running” has changed to the “rest”), the processor 110 may also determine that “exercise in which the pulse rate has been measured is finished” and determine the termination of the correction processing.

When the processor 110 determines not to terminate the correction processing (step S409; No), the processor 110 returns to step S405. When the processor 110 determines to terminate the correction processing (step S409; Yes), the processor 110 terminates the pulse rate correction processing.

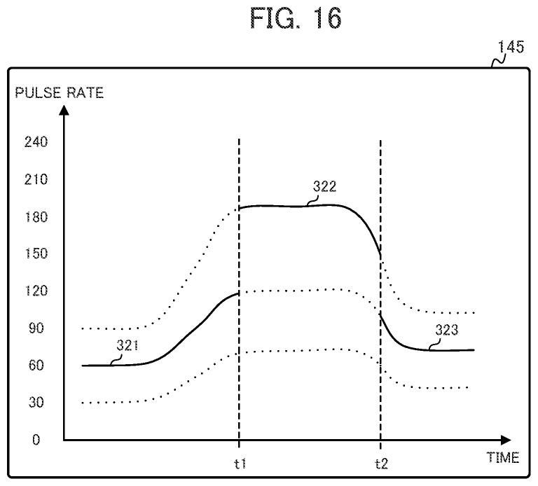

When, in the above-described case where the pulse rates illustrated in FIG. 14 are acquired, the pulse rate correction processing is executed and necessity of correction (the correct pulse rates are not the solid line 312 but the dotted line 311) is instructed to the electronic device 100 at the time t1, the pulse rates that were displayed by the dotted line 311 in FIG. 14 are displayed by a solid line 322 at and after the time t1, as illustrated in FIG. 16. In the examples in FIGS. 14 and 16, since, at and after a time t2, the correction error becomes considerably smaller than the correction error at the time t1 and the pulse rates with a likelihood of 40% come not to meet the conditions in step S406, it is configured such that, at and after the time t2, values indicated by the solid line 312 (a solid line 323 in FIG. 16) are displayed as correct values.

Although, in practice, the likelihood of a pulse rate has various values every time the pulse rate is calculated and it is often difficult to draw a graph by connecting pulse rates with the same likelihood, graphs each of which connects pulse rates with the same likelihoods with a line are illustrated in FIGS. 14 and 16 for facilitating understanding of the description. Note that, in the pulse rate correction processing, since it is possible to determine whether or not a pulse rate can be corrected depending on whether or not a pulse rate meeting the conditions in step S406 exists (that is, no pulse rate having the same likelihood needs to exist), it is possible to perform the correction processing without any difficulty even when a graph based on pulse rates having the same likelihood cannot be drawn.

In Variation 2, performing the pulse rate correction processing enables a more correct pulse rate after the correction to be displayed even when a pulse rate initially calculated is wrong.

Variation 3

Although, in the above-described embodiment and variations, the processor 110 selects a pulse rate measurement algorithm from among four types of algorithms, algorithms serving as selection candidates are not limited to the four types of algorithms. For example, the algorithms serving as selection candidates may be limited to two types of algorithms, namely the algorithm for low-level trend and the algorithm for high-level trend. As Variation 3, an embodiment in which a pulse rate measurement algorithm is selected from among the two types of algorithms is described.

In Variation 3, the processor 110 normally selects the algorithm for low-level trend as the pulse rate measurement algorithm. When the processor 110 estimates that the user has started exercise, the processor 110, estimating an increase in the pulse rate, selects the algorithm for high-level trend and, when the processor 110 subsequently estimates that the user has finished the exercise, the processor 110, estimating a decrease in the pulse rate, selects the algorithm for low-level trend.

Since, as described above, the algorithm for low-level trend outputs a moving average for a comparatively long time of the number of pulses as a pulse rate, frequency at which biometric detection values are acquired from the biometric detection value acquirer 130 can be reduced to a lower frequency than that at the time of selection of the other algorithms Therefore, the number of lightings of the LED of the biometric detection value acquirer 130 and the like can be reduced, as a result of which power consumption can also be reduced. On the other hand, since, in the algorithm for high-level trend, the algorithm for upward trend, and the algorithm for downward trend, it is required to acquire a moving average for a comparatively short time of the number of pulses, frequency at which biometric detection values are acquired is higher than that in the algorithm for low-level trend, as a result of which power consumption tends to be high.

In Variation 3, since narrowing down the algorithms to two types of algorithms enables not only a load of the algorithm selection processing to be reduced but also frequency at which the algorithm for low-level trend is selected to be increased to a higher frequency (than in the case where a pulse rate measurement algorithm is selected from among the four types of algorithms), power consumption of the electronic device 100 can be reduced.

Variation 4

In addition, the pulse rate measurement algorithms are not limited to algorithms matching change trends of pulse rates. Since the trend of biometric detection values detected by the biometric detection value acquirer 130 is supposed to differ depending on the type of exercise, it may be configured such that pulse rate measurement algorithms matching the types of exercises are prepared and, in the algorithm selection processing, the processor 110 estimates the type of exercise that the user performs and selects an algorithm matching the estimated exercise. For example, an algorithm used at the time of running, an algorithm used at the time of cycling using a bicycle, an algorithm used at the time of swimming, an algorithm used at the time of climbing a mountain, and the like are conceivable.

In addition, since, at the time of swimming, the trend of biometric detection values detected by the biometric detection value acquirer 130 is also supposed to differ depending on the style of swimming, it may also be configured such that an algorithm used at the time of swimming in the crawl style, an algorithm used at the time of swimming in the breaststroke style, an algorithm used at the time of swimming in the backstroke style, an algorithm used at the time of swimming in the butterfly style, and the like are prepared and the processor 110 also estimates a swimming style at the time of swimming and selects an algorithm suitable for the estimated swimming style.

Variation 5

Although, in the above-described pulse rate correction processing, the processing of correcting a pulse rate that the processor 110 calculated to a correct pulse rate by selecting a candidate of the pulse rate from among a plurality of candidates of the pulse rate that one pulse rate measurement algorithm (for example, the algorithm for low-level trend) being selected at that time calculated, based on the likelihoods and correction widths of the candidates is performed, candidates of the pulse rate are not limited to pulse rates that one pulse rate measurement algorithm calculated. It may be configured to, by including pulse rates calculated using other pulse rate measurement algorithms (when the algorithm for low-level trend is selected, algorithms other than the algorithm for low-level trend, such as the algorithm for upward trend, the algorithm for high-level trend, and the algorithm for downward trend) in the candidates of the pulse rate, cause the user to select a correct value from among the candidates (or select a correct value, based on comparison with a pulse rate measured by the reference device). This configuration also enables selection timing of a pulse rate measurement algorithm to be corrected.

In addition, although, in the embodiments described above, the description was made assuming that the electronic device 100 estimates a change trend of pulse rates, based on an estimated action detail of the user and calculates a pulse rate, using a pulse rate measurement algorithm matching the estimated change trend, information that the electronic device 100 calculates is not limited to a pulse rate. For example, the biometric detection value acquirer 130 including a sensor for measuring blood pressure enables the electronic device 100 to measure blood pressure of the user and also estimate a change trend of the blood pressure, based on an action detail of the user (for example, when the user is resting, the blood pressure is in a downward trend, and, when the user exercises, the blood pressure turns to an upward trend). Therefore, the electronic device 100 may measure blood pressure of the user, using an algorithm matching a change trend of the blood pressure.

In addition, without being limited to blood pressure, the electronic device 100 may, by increasing and decreasing types of sensors provided to the biometric detection value acquirer 130 as needed, measure any type of biometric information acquirable by the biometric detection value acquirer 130, using an algorithm matching a change trend of the biometric information.

In addition, although the pulse rate, the blood pressure, and the like, which are calculated based on information (biometric detection values) from the biometric detection value acquirer 130, are biometric information, it can be said that various types of information calculable from such information (such as a stress level and vascular age) are also biometric information. The electronic device 100 may measure any type of biometric information among the above-described types of biometric information, using an algorithm matching a change trend of the biometric information.

In addition, the electronic device 100 does not necessarily have to output biometric information in a manner of displaying the biometric information on the display. The electronic device 100 may output biometric information, for example, by voice.

Note that the electronic device 100 can also be achieved by a wearable computer that the user can wear on the body or a computer, such as a smartphone, a tablet, and a PC, that can acquire biometric detection values that sensors worn on the body of the user detected. Specifically, in the above-described embodiment, the description was made assuming that programs, such as a program executing the pulse rate display processing, that the electronic device 100 executes are stored in the storage 120 in advance. However, a computer that can execute the above-described processing may be configured by storing programs in a computer-readable recording medium, such as a flexible disk, a compact disc read only memory (CD-ROM), a digital versatile disc (DVD), a magneto-optical (MO) disc, a memory card, and a USB memory, and distributing the recording medium, and reading and installing the programs in the computer.

Further, it is possible to superimpose the programs on a carrier wave and apply the programs via a communication medium, such as the Internet. For example, the programs may be posted on a bulletin board system (BBS) on a communication network and distributed via the BBS. It may be configured such that starting up and executing the distributed programs in a similar manner to other application programs under the control of the operating system (OS) enables the above-described processing to be executed.

In addition, the processor 110 may be configured not only by any type of processor, such as a single processor, multiple processors, and a multi-core processor, alone but also by combining such any type of processor and a processing circuit, such as an application specific integrated circuit (ASIC) and a field-programmable gate array (FPGA).

In addition, although the description was made assuming that the processor 110 has a capability of executing the multi-thread processing, the multi-thread processing is not limited to parallel processing using multiple cores. Even when the processor 110 is configured by a single-core processor, the processor 110 may execute the respective pieces of processing in parallel by time-division processing by software, such as periodically performing the algorithm selection processing during the pulse rate display processing. In addition, the processor 110 does not have to have a capability of executing the multi-thread processing, and the processor 110 may execute the respective pieces of processing, for example, by a method of executing the algorithm selection processing every time at the end of the respective loops of the pulse rate display processing.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

What is claimed is:

1. An electronic device comprising:
a biometric detection value acquirer to acquire a biometric detection value for calculation of biometric information of a wearer of the electronic device; and
a processor,
wherein the processor
estimates a change trend of information related to an action detail of the wearer, and
based on the estimated change trend of information related to an action detail, selects an algorithm to calculate the biometric information from the biometric detection value.

2. The electronic device according to claim 1, wherein
the processor
estimates an action detail of the wearer,
based on the estimated action detail, estimates a change trend of the biometric information as a change trend of information related to the action detail, and
based on the estimated change trend of the biometric information, selects an algorithm to calculate the biometric information from the biometric detection value.

3. The electronic device according to claim 2, wherein
the processor,
in a case where an algorithm for low-level trend has been selected as the algorithm,
when the estimated action detail is exercise start, estimates an upward trend as a change trend of information related to the action detail and selects an algorithm for upward trend as the algorithm.

4. The electronic device according to claim 2, wherein
the processor,
in a case where an algorithm for upward trend has been selected as the algorithm,
when the estimated action detail is exercise end, estimates a downward trend as a change trend of information related to the action detail and selects an algorithm for downward trend as the algorithm, and
when the biometric information is greater than or equal to a high-level standard value and variation of the biometric information is less than or equal to a standard variation value, estimates a high-level trend as a change trend of information related to the action detail and selects an algorithm for high-level trend as the algorithm.

5. The electronic device according to claim 2, wherein
the processor,
in a case where an algorithm for downward trend has been selected as the algorithm,
when the estimated action detail is exercise start, estimates an upward trend as a change trend of information related to the action detail and selects an algorithm for upward trend as the algorithm, and
when the biometric information is less than or equal to a low-level standard value and variation of the biometric information is less than or equal to a standard variation value, estimates a low-level trend as a change trend of information related to the action detail and selects an algorithm for low-level trend as the algorithm.

6. The electronic device according to claim 2, wherein
the processor,
in a case where an algorithm for high-level trend has been selected as the algorithm,
when the estimated action detail is exercise end, estimates a downward trend as a change trend of information related to the action detail and selects an algorithm for downward trend as the algorithm, and
when, based on the estimated action detail, determining that exercise intensity increases, estimates an upward trend as a change trend of information related to the action detail and selects an algorithm for upward trend as the algorithm.

7. The electronic device according to claim 2 further comprising:
an exercise detector to acquire an exercise detection value relating to an exercise state of the wearer; and
an action history storage to store a past action detail of the wearer as an action history,
wherein the processor,
based on an exercise detection value acquired by the exercise detector and an action history stored in the action history storage, estimates an action detail of the wearer, and
causes the action history storage to store the estimated action detail.

8. The electronic device according to claim 1 further comprising an exercise detector to acquire an exercise detection value relating to an exercise state of the wearer,
wherein the processor,
based on an exercise detection value acquired by the exercise detector, estimates a change trend of information related to an action detail of the wearer.

9. A method for selecting an algorithm in an electronic device including a biometric detection value acquirer to acquire a biometric detection value for calculation of biometric information of a wearer and a processor, the method comprising
the processor:
estimating a change trend of information related to an action detail of the wearer; and
based on the estimated change trend of information related to an action detail, selecting an algorithm to calculate the biometric information from the biometric detection value.

10. The method for selecting an algorithm according to claim 9, wherein
the processor
estimates an action detail of the wearer,
based on the estimated action detail, estimates a change trend of the biometric information as a change trend of information related to the action detail, and
based on the estimated change trend of the biometric information, selects an algorithm to calculate the biometric information from the biometric detection value.

11. The method for selecting an algorithm according to claim 10, wherein
the processor,
in a case where an algorithm for low-level trend has been selected as the algorithm,
when the estimated action detail is exercise start, estimates an upward trend as a change trend of information related to the action detail and selects an algorithm for upward trend as the algorithm.

12. The method for selecting an algorithm according to claim 10, wherein
the processor,
in a case where an algorithm for upward trend has been selected as the algorithm,
when the estimated action detail is exercise end, estimates a downward trend as a change trend of information related to the action detail and selects an algorithm for downward trend as the algorithm, and
when the biometric information is greater than or equal to a high-level standard value and variation of the biometric information is less than or equal to a standard variation value, estimates a high-level trend as a change trend of information related to the action detail and selects an algorithm for high-level trend as the algorithm.

13. The method for selecting an algorithm according to claim 10, wherein
the processor,
in a case where an algorithm for downward trend has been selected as the algorithm,
when the estimated action detail is exercise start, estimates an upward trend as a change trend of information related to the action detail and selects an algorithm for upward trend as the algorithm, and
when the biometric information is less than or equal to a low-level standard value and variation of the biometric information is less than or equal to a standard variation value, estimates a low-level trend as a change trend of information related to the action detail and selects an algorithm for low-level trend as the algorithm.

14. The method for selecting an algorithm according to claim 10, wherein
the processor,
in a case where an algorithm for high-level trend has been selected as the algorithm,
when the estimated action detail is exercise end, estimates a downward trend as a change trend of information related to the action detail and selects an algorithm for downward trend as the algorithm, and
when, based on the estimated action detail, determining that exercise intensity increases, estimates an upward trend as a change trend of information related to the action detail and selects an algorithm for upward trend as the algorithm.

15. A non-transitory computer-readable recording medium storing a program executable by a processor of an electronic device, the electronic device including a biometric detection value acquirer to acquire a biometric detection value for calculation of biometric information of a wearer and the processor,
wherein the processor, in accordance with the program,
estimates a change trend of information related to an action detail of the wearer, and
based on the estimated change trend of information related to an action detail, selects an algorithm to calculate the biometric information from the biometric detection value.

16. The recording medium according to claim 15, wherein
the processor
estimates an action detail of the wearer,
based on the estimated action detail, estimates a change trend of the biometric information as a change trend of information related to the action detail, and
based on the estimated change trend of the biometric information, selects an algorithm to calculate the biometric information from the biometric detection value.

17. The recording medium according to claim 16, wherein
the processor,
in a case where an algorithm for low-level trend has been selected as the algorithm,
when the estimated action detail is exercise start, estimates an upward trend as a change trend of information related to the action detail and selects an algorithm for upward trend as the algorithm.

18. The recording medium according to claim 16, wherein
the processor,
in a case where an algorithm for upward trend has been selected as the algorithm,
when the estimated action detail is exercise end, estimates a downward trend as a change trend of information related to the action detail and selects an algorithm for downward trend as the algorithm, and
when the biometric information is greater than or equal to a high-level standard value and variation of the biometric information is less than or equal to a standard variation value, estimates a high-level trend as a change trend of information related to the action detail and selects an algorithm for high-level trend as the algorithm.

19. The recording medium according to claim 16, wherein
the processor,
in a case where an algorithm for downward trend has been selected as the algorithm,
when the estimated action detail is exercise start, estimates an upward trend as a change trend of information related to the action detail and selects an algorithm for upward trend as the algorithm, and
when the biometric information is less than or equal to a low-level standard value and variation of the biometric information is less than or equal to a standard variation value, estimates a low-level trend as a change trend of information related to the action detail and selects an algorithm for low-level trend as the algorithm.

20. The recording medium according to claim 16, wherein
the processor,
in a case where an algorithm for high-level trend has been selected as the algorithm,
when the estimated action detail is exercise end, estimates a downward trend as a change trend of information related to the action detail and selects an algorithm for downward trend as the algorithm, and
when, based on the estimated action detail, determining that exercise intensity increases, estimates an upward trend as a change trend of information related to the action detail and selects an algorithm for upward trend as the algorithm.
